(19) United States
(12) Patent Application Publication
(10) Pub. No.: US 2012/0157794 A1
(43) Pub. Date: Jun. 21, 2012

(54) SYSTEM AND METHOD FOR AN AIRFLOW SYSTEM

(76) Inventors: Robert Goodwin, Mercer Island, WA (US); Erik Jansen, Mercer Island, WA (US); Mark Tuacher, Bath (GB); James West, Bristol (GB); David Burgett, Newman Lake, WA (US); Jeff Anderson, Medical Lake, WA (US); Duane Radmar, Cheney, WA (US)

(21) Appl. No.: 12/973,863

(22) Filed: Dec. 20, 2010

(51) Int. Cl. A61B 5/0205; A61B 5/01; A61B 5/08; A61B 5/145; A61M 16/00; A61M 16/10

(52) U.S. Cl. 600/301; 128/207.18; 128/203.12

(57) ABSTRACT

Methods, apparatus, and systems for an airflow system, wherein the positive applied pressure is maintained at an approximately constant level over a variety of conditions. In one embodiment, a laminar flow is maintained by a foam housing and configuration. The use low air volume is enabled by using a closed system, wherein feedback information from the system is used to control the speed of an impeller. In one embodiment, moisture recirculation and recycling is applied to extract moisture from exhalation and inject the moisture back into the inhalation portion of the breathing cycle.

### CLAIM OF PRIORITY

This application claims priority to the following applications:
“Method and Apparatus for an Apnometer Device,” having Attorney Docket No. 2010.002US1, and U.S. application Ser. No. ______ filed Feb. 26, 2010, and
System and Method for an Airflow System, having Attorney Docket No. 2009.001US1, and U.S. application Ser. No. ______, filed Dec. 21, 2009,
each of which is incorporated by reference.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent tiles or records, but otherwise reserves all copyright rights whatsoever. The following notice applies to the software and data as described below and in the drawings that form a part of this document: Copyright 2009 Goodwin Industries. All Rights Reserved.

### BACKGROUND

Airflow systems are used for respiratory therapy so as to improve breathing function, such as for sleep apnea, hypopnea, snoring, and other respiratory related conditions. One example of an airflow system is a Continuous Positive Airway Pressure (CPAP) system, which is used to improve oxygenation in spontaneous breathing as well as in ventilation systems. Even with the use of a CPAP system, a user may experience respiratory disruptions.

### BRIEF DESCRIPTION OF THE DRAWINGS

FIG. 1 is a block diagram illustrating an airflow system, according to an example embodiment;

FIG. 2 is a flow diagram illustrating operation of controlling an impeller according to an example embodiment;

FIG. 3 is a block diagram illustrating an airflow system according to an example embodiment:

FIG. 4 illustrates various views of an airflow device, according to an example embodiment.

FIG. 5 is a detailed view of an airflow device, according to an example embodiment.

FIG. 6 is a detailed view of an impeller and splitter, according to an example embodiment.

FIG. 7 is a view of a portion of a nasal attachment, according to an example embodiment.

FIG. 8 is a block diagram of an Integrated Circuit (IC) which is part of an airflow device, according to an example embodiment.

FIGS. 9A-9H illustrate various views of a cleaning unit for an airflow device, according to an example embodiment.

FIG. 10 is a headband with a battery pack for an airflow device, according to an example embodiment.

FIG. 11 is an airflow device with moisture recycling components, according to an example embodiment.

FIGS. 12 and 13 illustrate two views of a user wearing an airflow device according to an example embodiment.

FIG. 14 illustrates an apnometer, according to an example embodiment;

FIGS. 15 and 16 are flow diagrams illustrating operation of the apnometer of FIG. 14, according to an example embodiment.

FIG. 17 illustrates a wireless device incorporating methods for analyzing sleep apnea criteria, according to an example embodiment.

FIG. 18 is a timing drawing illustrating a sample window for analyzing sleep apnea criteria, according to an example embodiment.

### DETAILED DESCRIPTION

In the following description, for purposes of explanation, numerous specific details are set forth in order to provide a thorough understanding of some example embodiments. It may he evident, however, to one of ordinary skill in the art that embodiments of the invention may be practiced without these specific details.

There are a variety of physical conditions that may result in a need for a person to require assistance in keeping their airway open to ensure proper breathing during sleep. One condition is called “apnea” or sleep apnea, and describes scenarios where muscles relax causing the airway to collapse reducing airflow and thus oxygen to the brain. Various treatments have developed to aid sufferers of apnea, including the use of a pressurized airflow device to keep the airway open.

A CPAP device is one example of an airflow device which provides a stream of pressurized air through a mask to a user. The pressure applied by the airflow keeps the airway clear or obstructions, resists relaxation of the muscles supporting the airway, and thus allows normal breathing during sleep.

A typical CPAP device includes a compressor unit, a length of tubing, and a facial connection mechanism, which may be a facial mask, similar to an oxygen mask, or a nasal pillow(s) that presses up against the bottom of a user's nose. The CPAP device is set up by a specialist, or physician who adjusts the airflow and adjusts the device for the patient. The CPAP settings are generally calibrated by a specialist or ear-nose-and-throat physician during a sleep study. This process causes the patient considerable inconvenience both in time and privacy.

Current CPAP devices are cumbersome and can be ineffective. In some instances the airflow is provided in a manner that causes the nose to dry out, requiring the user to additionally acid a humidifier to humidify the air. This double processing adds complications to the CPAP system, as the humidifier may add water build-up in the user, such as in the sinus or ears.

Additionally, traditional airflow systems require customization to each user, wherein each unit and breathing interlace apparatus is personally adjusted to each user. This adjustment adds to the variability of the experience and results of the airflow generator.

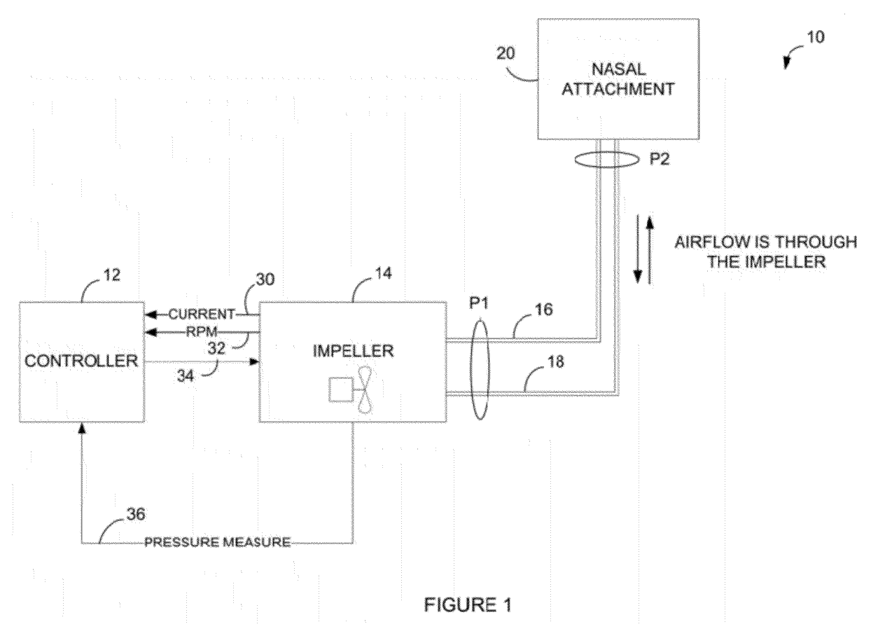

FIG. 1 illustrates an airflow system according to an example embodiment. The system 10 includes an impeller 14 for generating airflow to a nasal attachment 20. The airflow is provided from the impeller 14 through connectors, such as tubes 16, 18, to the nasal attachment 20. In some embodiments, the exhaust air is expired through the impeller to the atmosphere. The impeller 14 provides the airflow defined by several parameters, including: i) flow rate T, which may be measured in liters per minute (LPM), applied positive pressure P_{APPLIED}, and a pressure set point P_{SET}. The applied positive pressure may be measured at point P1 proximate the output of the impeller 14 or at point P2 proximate the input to the nasal attachment 20. The term impeller is used to refer to a device providing airflow having such parameters (T, P_{APPLIED}, P_{SET})

As the user breathes out, exhaust is expelled through the tubes 16, 18 to the impeller 14 and out the impeller 14 and into the atmosphere. There may also be some carbon dioxide, CO2, buildup in the airflow device due to a small volume of gas in the hoses 16, 18 and the impeller 14 allowing CO2 to accumulate. This volume, however, is small enough to avoid excessive CO2 buildup and thus avoids potential medical concerns.

In one example, the nasal attachment 20 is designed to correspond to the shape and orientation of the entry into the nasal cavity, which is further detailed hereinbelow. The nasal attachment 20 may include two units, one to be positioned within each nostril, wherein each of the units of the nasal attachment 20 has an opening consistent with a size of a nasal opening, but without a reservoir or waste gate. The nasal attachment is configured to receive the airflow directly from the impeller 14 with minimal interference. The configuration of system 10 provides a smooth continuum for the airflow as generated from the impeller 14, to flow through the tubes 16, 18 and through the nasal attachment 20 into the nasal passages of the user. When air flows over a smooth consistent surface the flow is less turbulent and may be maintained as a laminar flow. In this way, system 10 is designed to avoid turbulent airflow behavior.

The impeller 14 is further coupled to a controller 12 which controls operation of the impeller 14. The controller 12 receives operational information about the impeller 14, such as speed of the engine, Rotations per Minute (RPM) 32, and current 30. From this information the controller 12 determines whether an adjustment is to be made to the impeller 14 and if so sends a control message or signal 34. The controller 12 maintains constant pressure. The system measures the pressure and then uses the pressure measurement to adjust the current supplied to the motor of the impeller 14, or to adjust the RPM of the motor, and thus implement control of the impeller 14. Voltage adjustments are made to the motor to respond to changes in pressure. For example, when the current measurement may indicate that the speed of the impeller may he decreased or increased, the controller 12 will indicate such a change to the impeller 14. Another indicator or combination of indicators may be used to identify a pressure condition of the system 10, which is then used by the controller 12 to adjust the positive applied pressure P_{APPLIED} generated by the impeller 14.

The controller 12 may be implemented in software or hardware, or as a combination of both. In one embodiment, an Application Specific Integrated Circuit (ASIC) is designed to respond to changes in current and pressure according to a predetermined scheme, such as through use of a microcontroller and software to control the impeller operation. In another embodiment, firmware acts in coordination with hardware to adapt to changes quickly. In some embodiments, software is used to track behavior of the impeller 14, and a historical record is maintained so as to respond quickly to changes and to anticipate the behavior of the user.

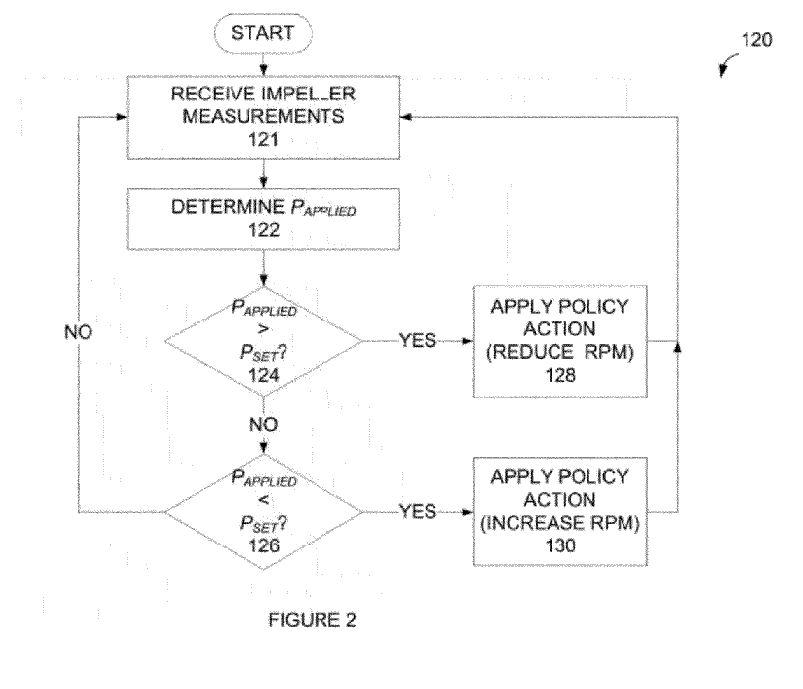

FIG. 2 is a flow diagram illustrating operation of controlling an impeller according to an example embodiment. The method 120 starts when pressure information is received at a controller 12 that indicates the positive applied pressure P_{APPLIED} , such as measured as position P1, operation 121. The controller 12 then determines a value of P_{APPLIED} from the measurements, operation 122. The pressure information may include a current measurement from the impeller 14 that corresponds to the positive applied pressure P_{APPLIED}, or may include a rotational speed or RPM of the impeller 14.

In one embodiment a feedback loop is implemented to use the pressure information to make decisions for adjustment of the impeller 14 operation. This involves comparing a first pressure to the measured pressure to identify a difference or delta value, wherein the first pressure may he calculated or selected. The first pressure is effectively a target pressure that may be determined ahead of time by the airflow device manufacturer, or may he dynamically calculated during operation. The feedback operation may he implemented such that the integration over time of the difference or delta value (between the first pressure and the measured pressure) as an output. The output value is then used to control, for example, to raise or lower, the voltage of motor. In some embodiments, the use of the RPM and the current information may also be used to improve responsiveness of the system. For example, some systems may measure the RPM of the motor or the current input to the motor and use one or both as an input to the feedback control mechanism. Still further, some embodiments may consider a combination of the various parameters, airflow pressure and motor operational characteristics, to control impeller operation.

The controller 12 uses the pressure information to apply an adjustment policy which maintains the airflow at achieve a desired result, wherein the airflow is generated at a comfortable pressure for the user sufficient to maintain an open airway and to sustain a laminar flow. A variety of algorithms may be implemented to control the airflow. A variety of airflow policies may be implemented consistent with laminar airflow. The adjustment policy may he set according to a variety of criteria, including a user's comfort level or according to a medical or health criteria.

In one example, the adjustment policy allows an adjustable flow rate T while maintaining a constant positive applied pressure, which is in contrast to traditional approaches that maintain a constant flow rate and a constant pressure. The embodiment illustrated in FIG. 2 implements a feedback loop to receive information from the output of the impeller, which the controller 12 uses to adjust the speed of the impeller 14. In this way, the controller 12 adjusts the flow rate T by adapting the speed of the motor to operational conditions. This allows the system 10 to adapt to each user's specific physique and condition. The adjustment policy may adjust the speed of the motor driving the impeller 14 to respond or adapt to a user's breathing cycles. As a person sleeps, for example, the breathing conditions change, and by maintaining an approximately constant positive applied pressure of the generated airflow the controller 12 is able to adjust the airflow accordingly. In some embodiments a high torque motor is used to allow for rapid speed change of the motor so as to adapt quickly.

In the embodiment illustrated in FIG. 2 the adjustment policy compares the positive applied pressure P_{APPLIED} to a pressure set point P_{SET}. When the two values are approximately equal no action is taken and the operation is at target or goal. When the positive applied pressure P_{APPLIED} strays from the pressure set point P_{SET} the controller 12 adjusts the impeller 14 to return P_{APPLIED} to approximately P_{SET}. Note, in some embodiments the process does not require express consideration of the flow rate T. This is to avoid the difficulty and expense of such measurements. It is possible to determine or estimate the value of the flow rate T from the relationship:

T=f(impeller speed, impeller current, P_{APPLIED}) Equ. (2)

In this way, calculation, or determination, off the airflow T provides the potential for a faster response to variations in pressure. Generally, once the significance of the parameters is understood, this information may be used to better predict how variations in voltage will impact pressure. In some scenarios, one parameter may be the salient parameter influencing pressure changes and therefore it may be sufficient to use a single parameter as an input for such an adjustment policy. In some scenarios, a combination of parameters may be used for the feedback loop to implement an adjustment policy. Still further, there are some embodiments where the feedback parameters may be selected based on historical operation oldie airflow device, wherein operation is tracked to determine the result of previous actions taken. This also introduces complexity which may or may not be needed.

The method 120 checks at decision point 124 if the P_{APPLIED} is greater than the P_{SET}, and if so applies a policy action at operation 128, such as to reduce the speed of the impeller 14, which may be to reduce the RPM of the impeller motor. At decision point 126 if the P_{APPLIED} is less than the P_{SET}, the controller 12 applies a policy action at operation 130, such as to increase the speed of the impeller 14.

Some embodiments consider the combination of parameters (T, P_{APPLIED}, P_{SET}) in making the control decisions. In this way, the impeller 14 provides a measure of the flow rate T to the controller 12 in addition to the pressure indicators. Note, an indicator may provide information as to multiple parameters, such as where one indicator is used to identify the condition or value of pressure and flow rate.

Method 120 of FIG. 2 uses the pressure set point P_{SET} as the threshold or target value for adjustment and for implementing an adjustment policy. Typically, the pressure set point may be determined during an initialization stage, referred to as a learning stage, wherein the user first sets up the device. This may be an automatic procedure, wherein the device determines an optimum pressure to maintain both a laminar airflow as well as to achieve a comfort and respiratory relief level for the user. This may also he a manual process, wherein the user selects a setting that is comfortable based on actual use. In an example embodiment, a learning stage is not required, as the system is effectively a closed system with no leakage, and therefore, the settings may not need to he adjusted for each individual.

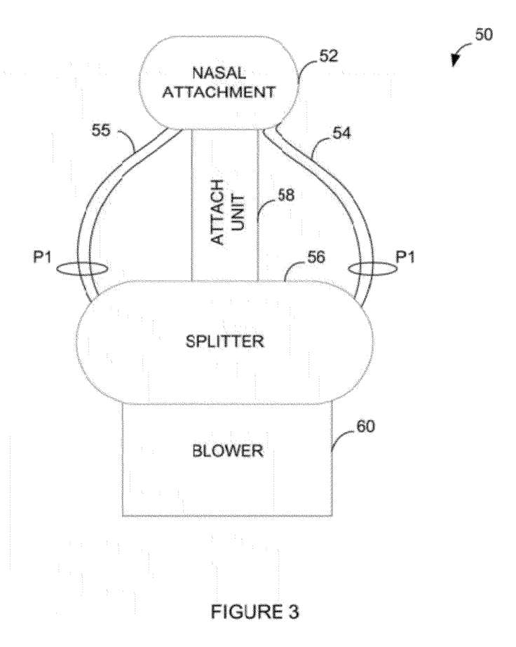

FIG. 3 illustrates an example embodiment of a system 50, similar to system 10, having an impeller 14 and controller 12 housed in a blower 60. The system 50 includes a splitter 56, an attach unit 54 and a nasal attachment 52. The attach unit 4 couples the nasal attachment 52 to the splitter 56. The splitter 56 provides the airflow from the blower 60 to each nostril of the user through connectors 54 and 55 to each unit of the nasal attachment 52. As illustrated, a measurement point of pressure PI is considered just proximate the blower 50. The connectors 54, 55 may be tubes or hoses and may be a made of a variety of materials so as to encourage a laminar air flow. The nasal attachment 52 includes gaskets to connect to each nostril.

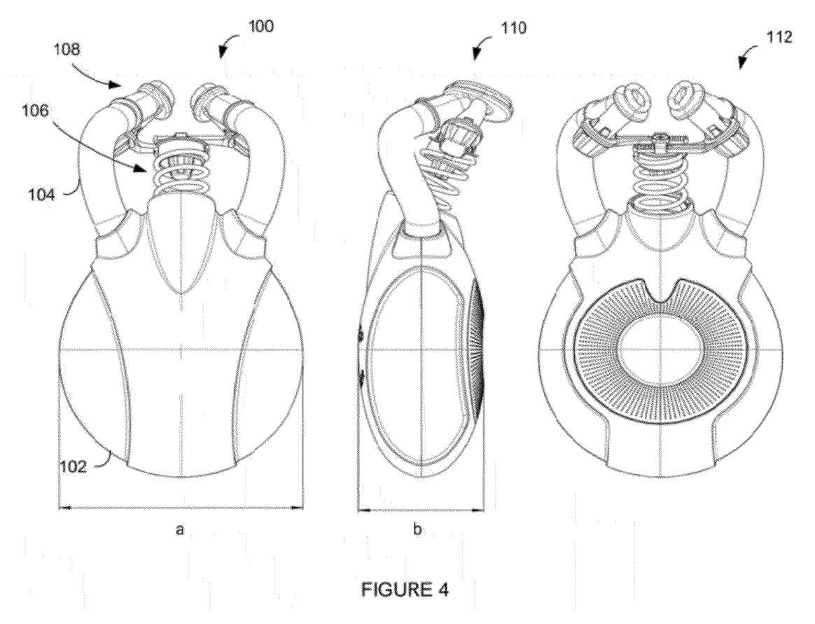

FIG. 4 is an embodiment of a system 100 similar to system 50, wherein the blower 60 and the splitter 56 are airflow unit 102. The airflow unit 102 is coupled to nasal attachment 108 with attach unit 106, which may be a spring or other flexible member. The nasal attachment 108 includes two units, one for each nostril, which are shaped to conform to the shape of the nasal cavity. The nasal attachment 108 couples to the airflow unit 102 with the attach unit 106 so as to allow movement and adjustment. In contrast to traditional approaches using a physician or technician to custom lit a CPAP device or apparatus to an individual's face, the embodiment of FIG. 4 does not require such customization. The system 100 accommodates to the variations in facial differences. In the illustrated embodiment, springs allow such accommodation as once in place, the system self-adjusts. In some embodiments a user may adjust a tension of the system 100 initially, after which the system 100 will self-adjust.

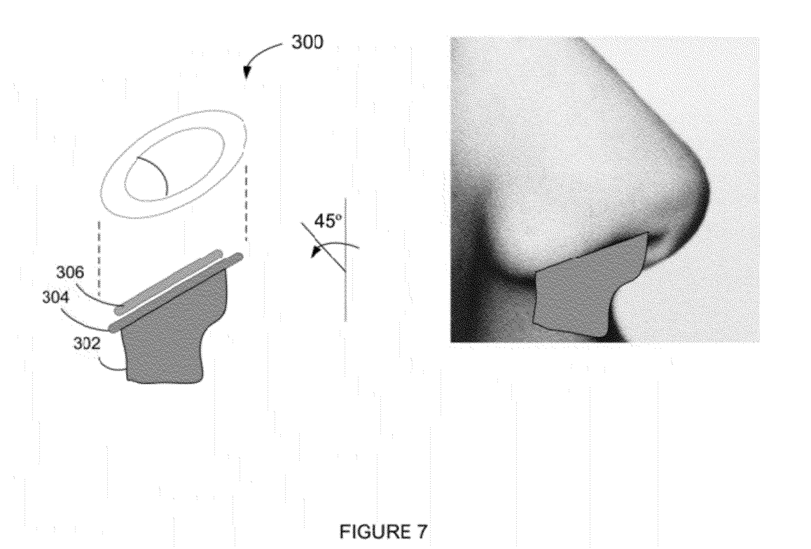

Each of the units making up the nasal attachment 108 includes a connector portion and a gasket portion. The gasket portion follows the flow of the nasal cavity rather than traditional CPAP devices wherein the nasal connector directs flow in a direction that is approximately perpendicular to the bottom of the nose. The nasal attachment 108 is further detailed in FIG. 7, hereinbelow.

FIG. 4 illustrates a front-view of the system 100 having a width a, and also includes other views of the system 100. View 110 is a side-view of the system 100, having a depth b, and further view 112 is a rear-view of the system 100.

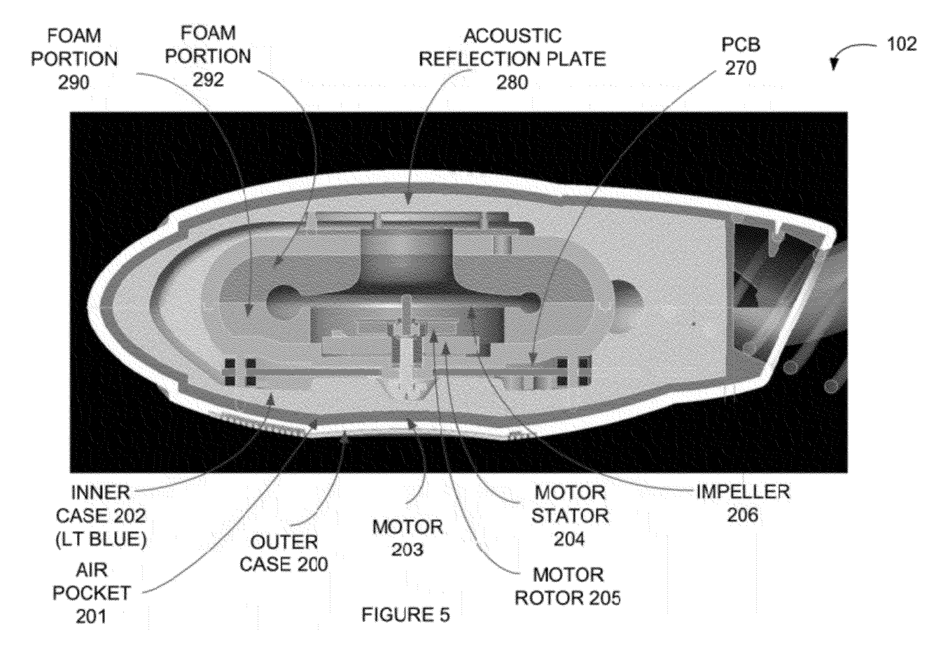

The airflow unit 102 is further detailed in FIG. 5. The airflow unit 102 has an outer case 200, which in one example is composed of rubber or other material to provide comfort with respect to thickness, grip and appearance. The outer case 200 surrounds a second case 202, which in one example is composed of foam, which both forms the shape of the airway and absorbs acoustic noise. The airflow unit 102 acts to suppress turbulent airflow and assists in maintaining a laminar airflow output from the airflow unit 102. As illustrated an air pocket 201 is formed between the outer case 200 and the second case 202. The air pocket 201 in one embodiment is made of plastic or other lightweight material. The airflow unit 102 also includes a motor 203 having a motor rotor 205 and a motor stator 204 to drive the impeller 206. A Printed Circuit Board (PCB) is positioned within the airflow unit 102 and proximate to the motor 203. The PCB 270 includes a controller, such as controller 12 of FIG. 2. The controller is adapted to receive operational information from the motor 203, the impeller 206, or the pressure sensor (not shown in FIG. 2), and apply an adjustment policy in response. The adjustment involves adjusting operation of the impeller 206. The adjustment policy according to one embodiment seeks to maintain a constant positive applied pressure P_{APPLIED} to generate the airflow. The adjustment policy therefore, controls the speed of the motor 203.

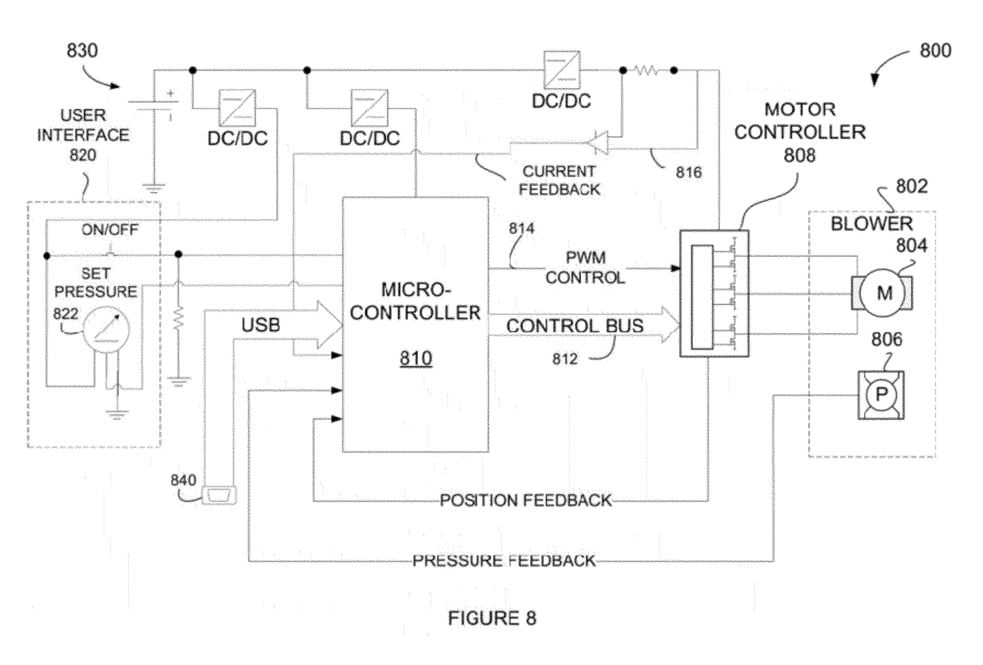

The PCB 270 and other portions of the airflow unit 102 are further described in FIG. 8, wherein unit 800 includes a microcontroller 810 to control operations of the impeller. Instructions and commands may he received from software, firmware or a user interface through USB 840, which may he a USB dongle or other removable memory device. The microcontroller also receives position feedback information from a motor controller 808, and pressure feedback hack information from a pressure sensor 806. The position information may further provide other motor parameters or characteristics, such as RPM, and so forth. Additionally, the microcontroller 810 receives current measurements or an indication of the power supplied to the motor. This information is provided from motor controller 808. The energy or power supplied to the motor is related to the speed of the motor, which is related to the pressure applied to the airflow, or positive airflow pressure. By transduction a such parameters and known or determined relationships, smart calculations and decisions may he made. This allows the airflow device to anticipate and react to changes quickly. The motor controller 808 controls the blower 802, or impeller, which includes the motor 804 and pressure sensor 806. The motor controller is fed from a power supply line 830. A current feedback loop 816 is also provided from the power supply line 830. Additionally a set pressure control 822 is provided to control a set point for pressure comparison with the applied positive pressure measured by pressure sensor 806. The set pressure value is provided to the microcontroller 810 which acts to compare this value to the measured pressure feedback information from the pressure sensor 806. Note, the pressure feedback information may he a pressure measurement or may he a coded value, such as an associated digital value. The unit 800 may be fabricated as a single IC unit, or may he built of multiple individual units.

The design of the airflow unit 102 in some embodiments is specific to maintain a laminar airflow over a variety of operating conditions. To this end, the airflow unit 102 includes a combination of foam portions 202,292 and 290 configured to minimize the introduction of obstructions, interference and other turbulence inducing constructs. An acoustic reflection plate 280 acts to reflect sound waves, effectively extending their flow path.

The resultant laminar flow generated within the airflow unit 102, which may also be referred to as a blower or a flow generator, adds to the efficiency of the air flow system. In many situations, the laminar flow near and proximate the nose adds to the comfort for the user, including a quitter operation and a reduction in the dryness experienced in the nasal cavity. Generally laminar flow is provided by manufacturing the airflow system with internal surfaces that are smooth with minimal transition points. This involves keeping angles small and gradual, maintaining nearly constant flow diameters and avoiding protrusions into the established flow path. For example, considering the system of FIG. 3, the connection between the nasal attachment 52 and each of the tubes 54, 55 may be manufactured as a single unit with a smooth connection, or may be designed as a snug fitting connection, so as to provide a minimal intrusion into the airflow paths. Further, the connections of the tubes 54. 55 to the splitter 56 are manufactured to minimize intrusion into the airflow paths. Finally, the connection of the splitter 56 to the blower 60 is also manufactured so as to minimize the intrusions into the airflow path. This is done by using materials that are smooth or encourage laminar airflow, and by shaping these paths to direct airflow without obstruction. Therefore, various embodiments implement a variety of designs, materials, shapes and mechanisms to reduce obstructions, protrusions, intrusions, interferences, and inconsistent surfaces, and to encourage laminar airflow.

Various materials may he used to build airflow systems to support a desired combination of some or all of the design goals, such as laminar airflow, efficiency. reduced noise, increased comfort, reduced nasal dryness, balanced thermodynamic operation, moisture control, and so forth. The inner surface of the airflow unit 102 is typically difficult to design for generation of laminar airflow, due to the shape, curvature and reduction zones associated with directing the airflow. Additionally, foam is typically a rough surfaced material that introduces turbulence and other interference to the airflow. To overcome these and other obstacles, some embodiments use smooth surfaced materials, such as shiny or polished walls. In an example embodiment, the airflow system is built by treating the open cell foam with a shiny surface layer to maintain an acoustic cell performance of the foam, while still affording the promotion of laminar flow. Further, as discussed hereinbelow with respect to hydrophilic materials, such a surfactant may act to avoid absorption of biological contaminants into the foam cells. In addition acoustic reflectors are added to reduce the turbulence of the airflow. The generation and maintenance of a laminar airflow provides a mechanism to reduce nasal dryness associated with traditional CPAP devices and without the use of a humidifier.

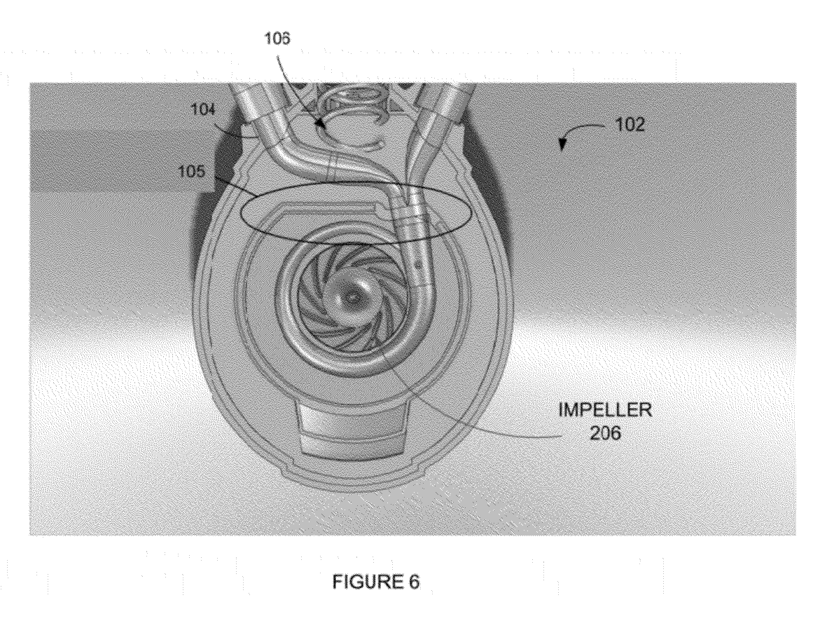

FIG. 6 illustrates another view of the airflow unit 102 having the impeller 206 in the center. A splitter 105 is used to evenly split the airflow and output it into two hoses 104. The splitter 105 is similar to the splitter 56 of FIG. 3. The attach unit 106 is illustrated as a spring device which allows for self-adjustment once positioned on a user's face. The use of a spring for the attach unit 106 allows comfort for a user during movement or body repositioning, such as during sleep. A wide range of movements are allowed as the user shifts position, so that the position of the blower 102 with respect to the user's nose may change dramatically while maintaining a continuous seal between the pressure on the nose assembly 108 and the user's nose. The spring mechanism maintains sufficient pressure to enable the continuous seal. The spring may he coated with a material that is comfortable for the user, such as foam or cloth which causes little friction when in contact with the user's skin. Alternate embodiments may implement other mechanisms having a spring constant or other mechanism that will track with movement and react to maintain the pressure so as to enable the continuous seal of the nasal attachment 108 to the user's nose without disruption of airflow.

The laminar airflow further provides noise control through dual acoustic resonators having separate hoses for each nostril, angular nasal entry and better nasal shape fitting to minimize constriction at entrance. The dual resonators in one embodiment are the inside casing and outside casings of the blower. The dual nose tubes are for nasal laminar flow. The splitter introduces turbulence into the airflow, and therefore the 2*L. (L.length) distance from the splitter to the nose is used to re-establish laminar flow before the air enters the nose. According to an example embodiment, the configuration has a blower with an output airflow split into two (2) hoses or tubes, one for each nostril, which are each then introduced to each nostril at an angle consistent with the nasal entry. This configuration minimizes constriction of airflow during travel from the impeller to the nose. The angle of the nasal attachment is further discussed with respect to FIG. 7 hereinbelow, and is designed to be an ergonomic solution in contrast to traditional face mask designs and nasal pillow designs, which tend to he approximately perpendicular to the base of the nose, parallel to the plane of the face. The example configuration designs a laminar flow, and therefore angles the nasal attachment as an approximate extension of the natural anatomy of the nose. Such a rhino-ergonomic design reduces constrictions and turns in the airflow path, encouraging laminar flow.

Passive noise reduction may be achieved through the use of material selections and physical layout of the airflow device. For example, the use of an inner resonator and an outer resonator provides for noise reduction and enhances the laminar feature of the airflow. One embodiment of a nasal attachment 300 is illustrated in FIG. 7 including a base portion 302 which couples to the hose, such as hose 104 of FIG. 5. The base portion 302 has a smooth internal surface that is configured to encourage laminar airflow. The nasal attachment 300 further includes an outer portion 304 and an inner unit 302. The outer portion 304 forms a seal at the nostril orifice, while the inner portion 302 follows the nasal cavity, fitting to the shape of the nose. The nasal attachment 300 may be a one piece unit or may he composed of multiple pieces. The nasal attachment 300 is designed to fit into the nose of the user and thus reduce or eliminate leakage of air during operation of the airflow system. The inner portion 302 may extend into the nasal cavity and is shaped to extend at an approximately 45° angle to from the vertical axis. This is to match the natural angle of the nasal opening into the sinus cavity which reduces leaks and enables a more secure contact between the nasal attachment 300 and the user's nose.

Some embodiments provide white noise or other additive noise to diminish the noise of the impeller. The airflow system may provide music, such as to allow a user to select music or to connect to an MP3 player or iPod.

The airway system may further have wireless capability to communicate with other systems, such as a medical information system. In one embodiment, the airway system stores information regarding the breathing cycles or patterns of the user and provides such information on request to a medical information system. On occurrence of a predetermined event, such as changes in breathing patterns or other event, the airway system contacts the medical information system.

There are a variety of ways to power an airflow device, some of which may he implemented within the airflow device positioned on the user's face. In one embodiment an integrated chin strap is attached to the airflow unit, such as airflow unit 102. The chin strap encourages the user to breathe through their nose and close their mouth, and thereby reduce leakage through the mouth. Some embodiments have improved performance using less air volume, such as in a configuration the airflow unit 102 contains the exhaust air, and does not have a waste gate, and has improved nasal seals. This allows a gentle impeller operation, reducing noise and improving laminar flow. In such a system, leakage through the mouth could overwhelm the system, and therefore it is desirable to encourage the user to breathe through their nose to the exclusion of their mouth. Breathing through a single orifice also results in a single valve point and thus keeping the mouth shut is a reliable way to keep the body's respiratory valve operating through the nose. When the chin strap is the primary mounting for the airflow unit, the chin strap may be used to keep the user's mouth closed. In one embodiment, the number of straps used is minimized so as to encourage full compliance by the user, and the airflow unit 102 is positioned below a discomfort zone on a user's face which may cause a claustrophobic reaction. The discomfort zone is typically close to the eyes, mouth and nose. Additionally the outlook is positioned so as to avoid the chest and provide a short, straight path to for the airway to the nose. The chin strap may further include springs to couple to the attach unit 104, which may also be a spring, so as to allow additional flexibility.

Maintaining a nasal seal reduces leakage and reduces noise and discomfort during sleep. Leakage may impact control of air pressure in the airflow system. In other words, increased leakage may add to the inefficiency of an airflow system. Head straps may be positioned between the nasal attachment 108 and the top of the user's head for further support and to maintain the nasal seal. This applies a force upward from the chin strap to the head piece. The use of springs then decouples unwanted forces when the user changes positions during sleep. In other words, movement which would normally act to displace the nasal attachment 108 is counter balanced by the chin strap and the strap over the head. The combination of positioned straps and springs provides a highly reliable, comfortable tit over a variety of positions and conditions for a variety of users.

The head strap may also use a head mounted battery pack, wherein the power supply is provided proximate the airflow unit 102. Various head strap configurations are considered, including adaptable, adjustable and other mechanisms.

In some embodiments, the user will breathe through the impeller, rather than having a waste gate, which maintains all of the air pressure within the system 10. As illustrated in FIGS. 3-5, in some embodiments the user breathes through the airflow unit 102. To avoid possible introduction of contaminants into the airflow unit 102 associated with some configurations or in some scenarios, leading to a potential requirement for periodic or daily disinfecting and cleanings. The design of the airflow unit 102 allows steam cleaning while the impeller 206 is operating. A cleaning unit may he operated when the system 100 is not in use by the user.

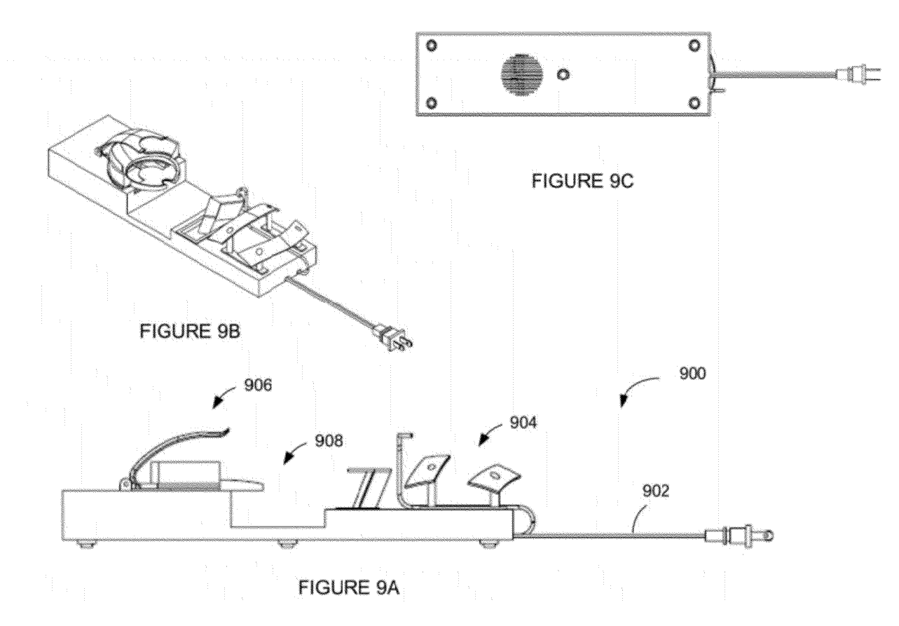

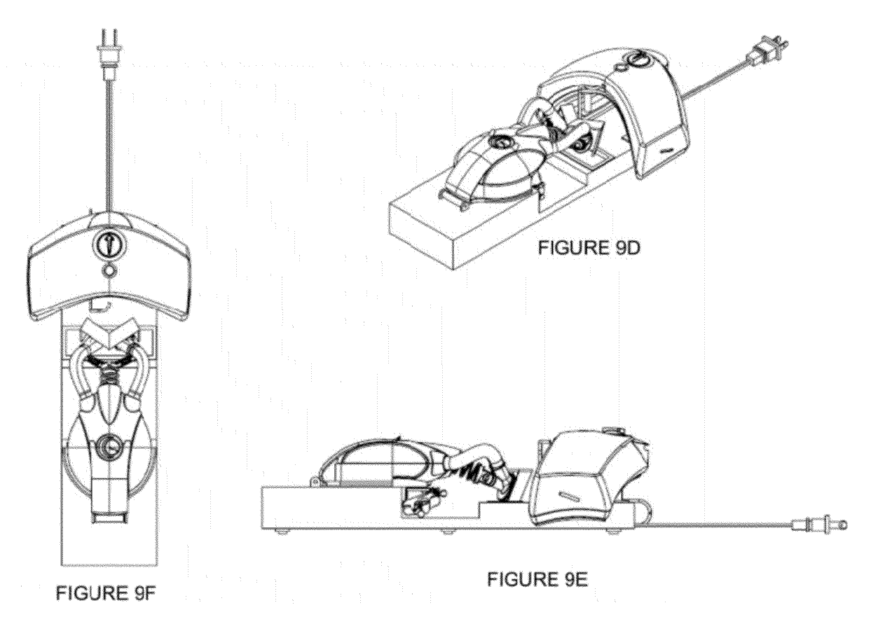

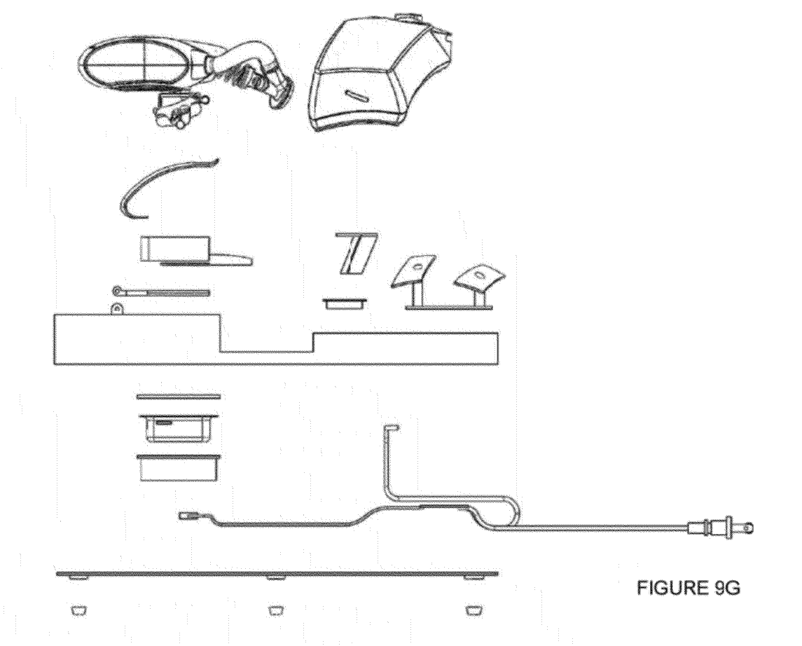

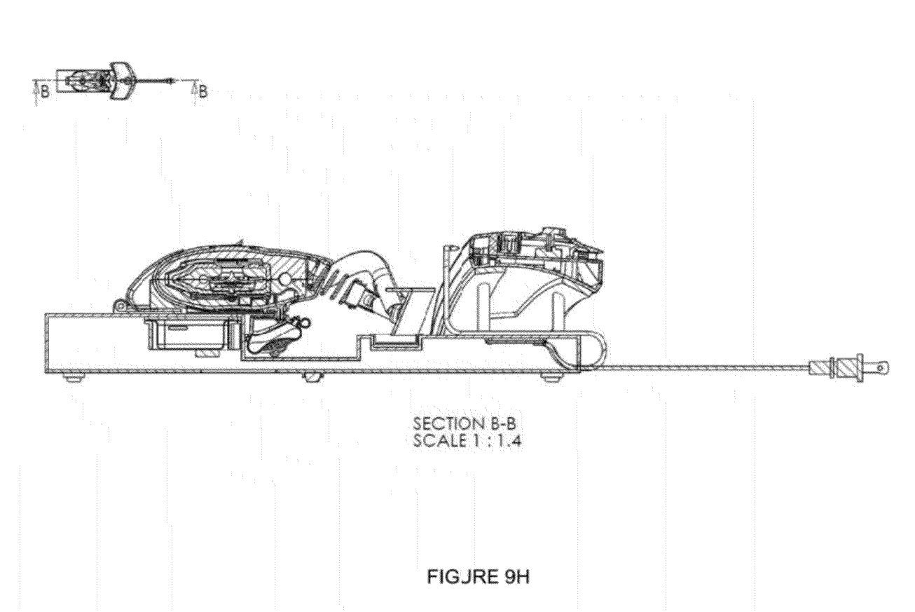

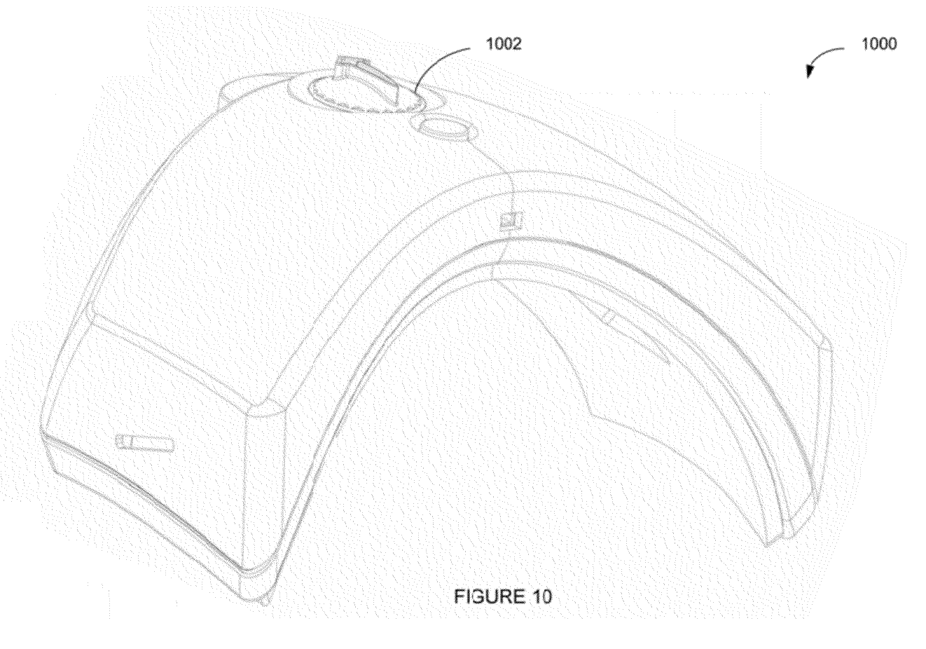

A cleaner system according to one embodiment is illustrated in FIGS. 9A-9E in various views. FIG. 9A illustrates a side view of the cleaner system 900 when empty. A top view is illustrated in FIG. 9B and a bottom view in FIG. 9C. The cleaner system 900 includes a charging portion 904 which is adapted to receive head band which includes a battery. The charging portion 904 is for charging the battery of the head band. The cleaner system 900 also includes a receptacle portion 908 for receiving the facial portion of the airflow device and a cleansing portion 906 which provides steam for cleaning the device. FIG. 9G illustrates the various components that form the system 900.

FIG. 10 illustrates a headband for operation with an airflow device. The headband 1000 supports a battery for powering the airflow device, and has a control 1002. The shape of the headband 1000 is configured to sit comfortably on the head and to provide support for the airflow device when positioned on the user's face straps (not shown) couple the headband 1000 to the airflow device, wherein various embodiments implement a variety of strap configurations and combinations. A design goal may he to minimize the number of straps to facilitate easy use. Other designs may consider a variety of facial dimensions or aspects to optimize comfort and fit, and therefore, may have an increased number of straps or securing mechanisms.

### Recycling Moisture

In another aspect, an airflow device, such as a CPAP device, provides a positive airflow to a user's airway in conjunction with moisture recycling, such as through the use of a hydrophilic material or an apparatus configuration, or mechanical mechanism. The ability to recycle moisture in an effectively closed cycle airflow system provides a more comfortable system for the user and avoids some of the problems intrinsic to conventional airflow devices, such as the use of additional humidifying hardware. The airflow system 10 of FIG. 1 is an effectively closed system as there is no specific waste gate, but rather, the user's exhaust is expelled through the impeller 14 as part of the blower exhaust. In such an airflow system 10, a method to recycle moisture on the exhalation portion of the breathing cycle, and then reintroduce that moisture during the inhalation portion of the breathing cycle, such as for use in respirators or in CPAP machines, provides moisture to the nasal passages avoiding drying.

In one embodiment, an airflow system captures humidity that is present in exhaled breath, and recombines it with the inhalation airflow thereby humidifying the next breath. In this way, the humidity is joined with the fresh air, or evaporated into the fresh air. During the exhalation of breath, air leaving the mouth or nose is relatively warm. In an ambient condition, a human exhalation is typically about 37° C. The exhaled air is also moist, being typically at about 95% Relative Humidity (RH). On exhalation, this air enters the ambient surroundings and quickly cools to match those conditions, which are typically cooler and dryer. As an example, ambient room temperature may he considered approximately 22° C. with 0-70% RH. As the exhaled air merges with the lower temperature, dryer ambient surrounding air, moisture in the exhaled air will condense. For example, it is expected that approximately 50% of the moisture in the exhaled air will condense onto a smooth surface in this situation. By providing an airflow device having a smooth airway chamber for exhalation flow, such as described hereinabove to achieve laminar airflow, a smooth surface is provided over which such moisture condensation may occur. In other words, a condensation surface is present in the airflow device having both distance and surface area sufficient to capture the moisture extracted from the exhaled breath. In addition, a natural temperature gradient is present along the airway since the air cools as it moves away from the nose. This temperature gradient spreads the moisture collection over the length of the tubing. In a situation where the exhaled air leaves the nasal attachment at near ambient temperature, and there is no leakage or escape near the nose, mouth, face or mask (if used), then only the saturated moisture content (100% RH) of the now cooler, ambient air will escape.

Upon inhalation, ambient air will enter the airway chamber (typically 22 C with 0-70% RH), and this air will pass by the same surfaces containing extracted moisture from the exhalation condensation This results in a wicking effect that will cause evaporation of the moisture into the air stream until the moisture is used up, or until the air has 100% RH. There will also be some stored heat in the chamber due to exposure to hotter exhaled air, so air will re-enter the nose or mouth at a temperature somewhat above ambient temperature, but with saturated moisture.

In some implementations the following are used as design factors, including an adsorbent surface material, sufficiency of the surface area, sufficiency of configuration and components to achieve acceptable re-evaporation of the moisture, and the material and configuration of the device. An adsorbent surface material on the exhalation chamber and flow surfaces may be designed to maximize the condensation and storage of water from exhaled breath on said surfaces. An adsorbent is a substance having a high surface area that can absorb substances onto its surface. Sufficient surface area in the condensation function may he designed to store the sufficiently condensed water. The size and type of extraction mechanism and reservoir, as well as the placement of such, impacts the efficiency and comfort of the device as well. Designs providing sufficient air speed, surface area, texture and surface chemistry may allow for complete re-evaporation of the water. In some embodiments a foam material with air pockets serves as an insulator for the storage of heat.

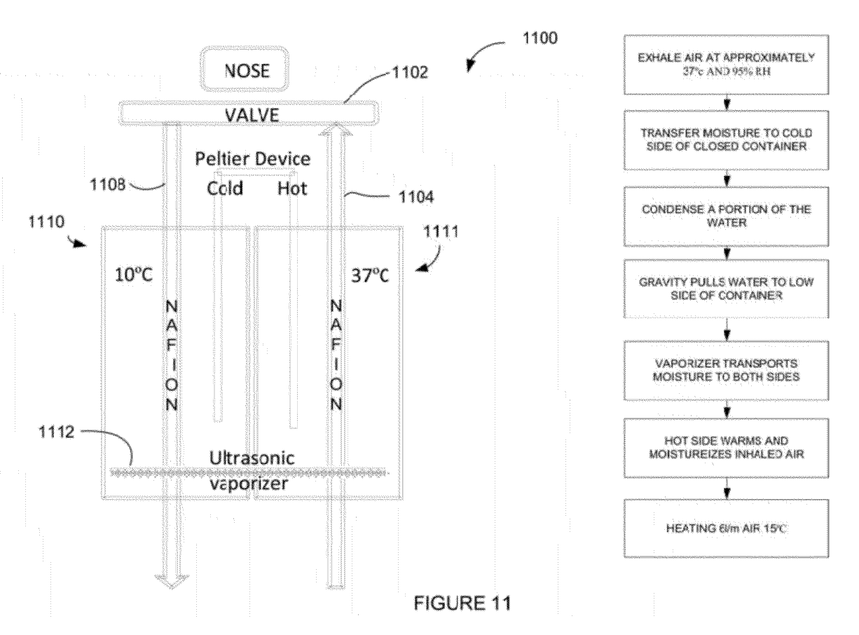

FIG. 11 illustrates, in a block diagram format, an example embodiment of airflow system 1100 implementing moisture recycling. As illustrated, the airflow device 1100 is positioned to form a seal with respect to the nose and includes tubes 1104, 1108, each made of a hydrophilic or other material which absorbs moisture out of the air as it passes through the system. A valve 1102 is configured between the tubes 1104, 1108 and the user's nose. A Peltier device having a first portion 1110, corresponding to a cold side of the airflow device 1100, and a second portion 1111 corresponding to a hot side of the airflow device 1100, is positioned so that the airflow goes through a vaporizer 1112, such as an ultrasonic vaporizer. In this way, the system captures the moisture of the air during exhalation and then uses this adds this moisture to the air on inhalation as vapor, and thus the vaporization processing of the inhalation air. In one embodiment, the hydrophilic material is NAFION material, which is a trademark of DuPont. The Peltier device actively cools the air during exhalation to create condensation; similarly the Peltier device actively heats the air on inhalation to create vaporization of the recycled moisture. The actions of the airflow device are similar to a nebulizer in dispersing the moisture in the airflow. This may be done ultrasonically, or with heating elements. In some embodiments one tube is used for inhalation and one tube is used for exhalation. In other embodiments, as single tube is used for both inhalation and exhalation, wherein the Peltier device oscillates quickly between increasing the temperature on inhalation and decreasing the temperature on exhalation.

Some embodiments implement solutions having a specified amount of water stored in the system and which is used by an ultrasonic nebulizer to cause evaporation. A steam cleaning process and system as described hereinabove with respect to FIGS. 9A-9G, may be used to recharge or maintain the small water container. Further, a vibrator or other mechanism may be used to atomize the water molecules, such as a small piezo-electric vibrator running at up to 1+MHz or more.

To better understand the benefit of recycled moisture processing, consider that a typical human body may exhale air at a temperature of 37° C. with a relative humidity of 95%, which is approximately 40 g/m3 of water in the air. Normal inhalation of ambient air typically has a moisture content measured from 0 to 20 g/m³ based on the relative humidity and temperature of the ambient air. For a given user in a given environment, these levels may change, and therefore, the pocket of warm moist air around the user's nose may deviate from these levels.

Many users experience nasal dryness when using airflow devices. It is common for CPAP devices and respirators to be used in combination with humidifiers to remedy this situation. Humidification increases the moisture content of the air delivered to the user during inhalation. In some examples, a water storage area is used to store water which is then converted to vapor, such as through heating or through ultrasonic excitation. Some examples require storage of sufficient amounts of water to maintain nasal moisture through eight (8) hours of usage; such storage containers are large and heavy, and therefore, impractical for face-mounting.

In one embodiment, a moisture recycling airflow device is face-mounted to capture the moisture from the air on exhalation, while providing a convenient, compact design for an airflow device. In another embodiment, a moisture recycling airflow device is not face-mounted, and may be used to overcome specifications and challenges associated with the condensation of water vapors as they move from a distant water storage area to the nose or mouth of the user. When a moisturizer is positioned far from the user, such as in use with conventional CPAP devices, the moisture can condense in the tubing and hoses connecting the CPAP device to the user's face mask, as these hoses remain cooler. There is further a need to prevent that “rain-out” in the hose, such as through the use of hose warmers in conventional devices. Moisture recycling voids these and other problems. These traditional moisturization solutions are independent of the breathing conditions and therefore are not sufficiently accurate. In contrast, by recycling the moisture as in an example embodiment, the moisture is a function of the breathing conditions and therefore provides moisturization proximate the user's nose.

In one embodiment, capturing the moisture during exhalation involves a separating the moisture from the exhaled air and storing the moisture in a reservoir, while still allowing the exhaled carbon dioxide, CO2, to escape into the atmosphere. The moisture capture stage is accomplished while the exhaled breath is warm and before the breath cools allowing the water vapor to condense. In one example, separation involves using a hydrophilic material, such as Nation Tubing at or near the connection to the nose or mouth. Water will pass through the wall of the tubing but oxygen, CO2, and Nitrogen will not. For water to transfer away from the exhaled breathe, there must be lower moisture on the other side of the tubing wall. For water to he transfer back into inhaled breathe, there must be higher moisture on the other side of the tubing wall.

In some embodiments, a passive system may simply apply a material or mechanism that employs the area on the outside of the tubing as a moisture reservoir. Inhaled air typically has less moisture, and is therefore “dryer,” than exhaled air. The moisture extracted from the exhalations is injected into the inhalation air stream. This processing recycles the moisture of the breathing cycle and increases the humidity of the inhalation. As approximately equal amounts of air are inhaled and exhaled, any reservoir of exhaled air is expected to eventually reach humidity half way between the exhale and ambient humidity levels. Inhaled air will therefore be moister than ambient air as the inhalations are now incorporating, moisture from previous exhalations. The moisture processing involves filtering through an antibacterial or other mechanism so as to maintain a germ-free and sufficiently purified environment, which may be designed and adapted according to use. For extremely clean environments, such as intensive care hospital use, this may involve highly sophisticated filtering, while average sleep apnea CPAP use may only require general small particle removal (not necessarily guaranteeing sterility).

As the user exhales, the moisture from the exhalation is extracted from the exhaled airflow and stored in the reservoir, wherein the reservoir may be configured in a variety of ways or utilized a variety of materials to accomplish the storage of the relatively small amounts of liquid moisture. In one embodiment, free air acts as the reservoir, wherein the extracted moisture is contained is a pocket of air, such as a balloon or mask. In another embodiment, a non-toxic chemical with strong moisture storage and transmission capabilities such as acrylamide sodium acrylate copolymer is used as a reservoir, wherein the reservoir is maintained in the immediate proximity of the flow generator and close to the nasal attachment for efficient operation due to the quick loss in temperature as the distance from the nose increases. Some embodiments may position the reservoir proximate the impeller or other places for convenience or cost savings. The reservoir may be made of a humidor gel type material. In some embodiments, the reservoir is a detachable member, such as a snap-on or a clip-on accessory to the body of the blower, such as under the side having the grilled intake facing the mouth and chin. In this sense, the reservoir is positioned for post-exhalation conditioning and pre-inhalation conditioning and is directly in the airflow path through the blower.

The reservoir acts in concert with the extraction mechanism so as to increase efficiency and reduce waste. In an active system, energy is used to transfer water to and from around the hydrophilic tubing to maximize the transfer of moisture to and from the breathed air during inhalation and exhalation. This concept involves maximizing the moisture around a common recycling tubing during inhalation, and minimizing the moisture during exhalation. This could involve more than one tube (one for inhalation and one for exhalation), but still achieves active transport of the water between inhalation and exhalation.

In one embodiment, a hydrophilic process cools the water vapor results in precipitation of water droplets for extraction, and then heats the water droplets into water vapor for moisturization. In some embodiments, the use of separate inhalation and exhalation tubes and/or reservoirs, such transfer would involve cooling the moist air in the exhalation chamber, condensing the water in a heating chamber, and then heating water so as to boil into a gas, which is then released into the inhalation chamber. In one embodiment, a single chamber system may he implemented that transfers water vapor to and from the single reservoir between inhalation and exhalation. Such transfer is approximately instantaneously, using a rapid cooling and heating process. Various other mechanism of active moisture exchange can be envisioned as well so as to extract moisture from the exhaled air and inject the extracted moisture into the inhaled air.

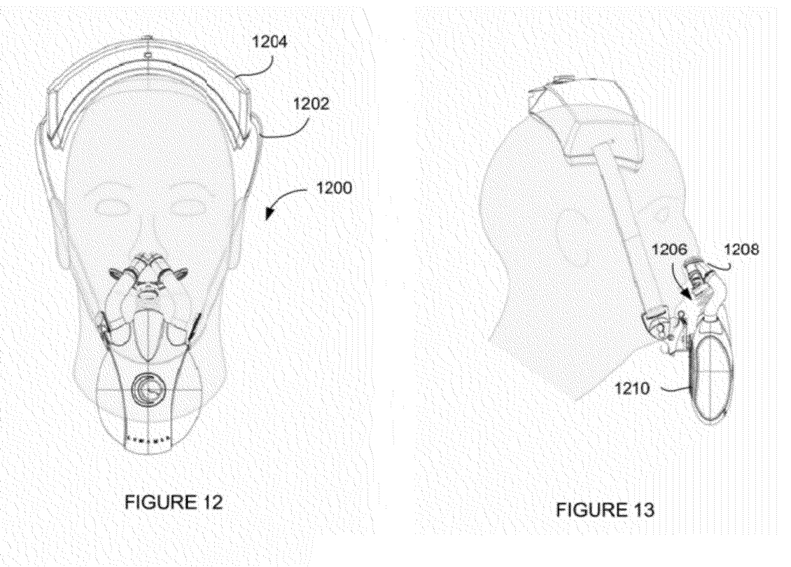

FIG. 12 illustrates a front view of a user wearing an airflow device according to an example embodiment. The airflow device 1200 has a headband 1202 and a battery pack 1204. FIG. 13 is a side view of the airflow device 1200. As illustrated, a minimal configuration of straps is implemented to maintain a seal between the nasal attachment portion of the airflow device 1200 and the user's nose. The airflow device 1200 further includes a flexible, responsive mechanism 1206, such as a spring, to connect the impeller portion 1210 to the nasal attachment portion 1208. A variety of other configurations are possible, however, the illustrated configuration avoids inconvenient positions for straps and belts in placing and maintaining the apparatus.

Such airflow systems often require customization to each user, wherein each unit is personally adjusted to each user. This adjustment adds to the variety of the experience and results of the airflow generator. A user may desire to understand their respiration over the course of an evening.

### Apnometer

The apparatus and techniques described hereinabove may benefit from the ability of the user to monitor respiration patterns, disruptions and events which are related to sleep conditions. Various monitoring techniques may be used to provide such information, however, these require either cumbersome machinery or intervention of a technician or physician. The user greatly benefits from the ability to monitor this information themselves. The feedback of such self-monitoring allows the user to maintain a record of their respiratory history and may he integrated with an airflow device to make adjustments in operation. Further, such historical information may be used by a physician in diagnosing and treating the user or patient.

In one embodiment, a device is provided to identify problems and conditions during sleep and provide a report. An apnea meter or “apnometer” is an integrated device which incorporates pulse-oximetry to detect and measure respiratory disruptions in a home setting. The apnometer identifies these disruptions and inconsistencies during sleep. This information may he used by a caretaker in determining a treatment plan, or adjustments to a current therapy. The apnometer integrates functions of detecting and summarizing respiratory dispruptions into a single device that attaches to the body. The apnometer may he designed to attach to a finger, toe, ear, nose, lip, cheek, chin, wrist, palm, and so forth. In some embodiments the apnometer may he adapted to a variety of locations, where the apnometer may auto adjust to the attachment location or a controller may be used to select the attachment location. The apnometer then provides a report of the detected respiratory information. While the present discussion considers a monitor placed on the linger, other scenarios may be implemented as well. As the measurements are performed using a Light Emitting Diode (LED) wherein the light shines through the skin, which identities the level of oxygenated blood through the detected color.

A Respiratory Disruption Index (RDI) is a measure of the respiratory disruptions in a given time period. The RDI may be used as an auxiliary diagnostic tool to help patients and doctors assess the presence of symptoms associated with sleep apnea. RDI is a metric widely accepted by the medical literature as a diagnostic tool for assessing sleep apnea.

Calculation of the RDI may use a variety of algorithms, such as by counting the number of respiratory disruptions identified during an 8 hour sleep period and then dividing this number by the number of hours of sleep. In one example, a respiratory disruption may be defined as an event lasting longer than 10 seconds where the user stops breathing and blood oxygen saturation falls 4 points below normal. Human scoring of RDI is generally required today, the RDI provides the physician information that identifies problem conditions and patterns for a given patient or user. The RDI is used to prevent discomfort and health issues related to the decrease in blood oxygen saturation, such as where the level falls below a tolerable level for the patient and may result in serious illness which is sometime fatal. For example, an RDI event may be identified as where the measured SpO2 falls 4 percentage points and then returns to the average. The average may be a fixed averaue used over a population of users, or may he an individually calculated average for a given user.

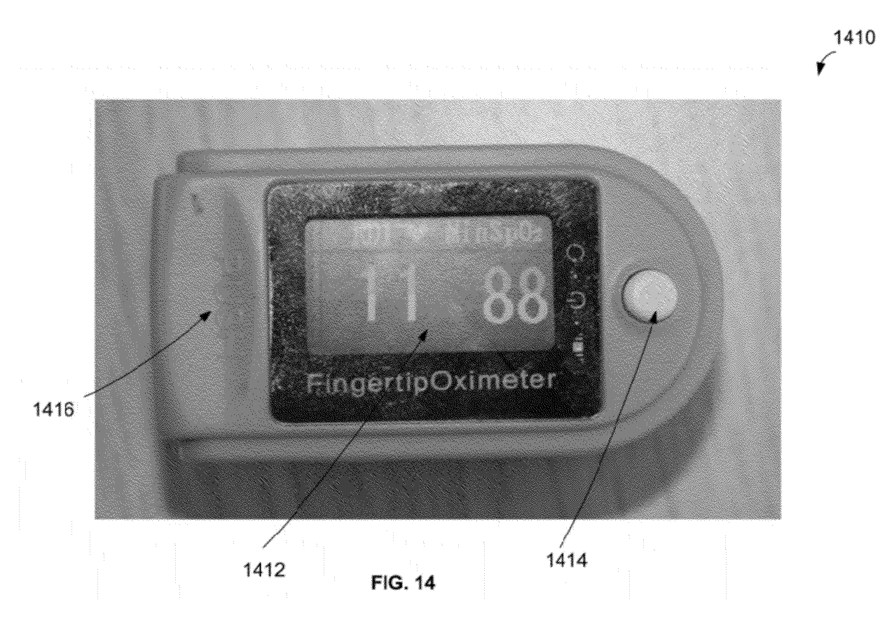

FIG. 14 illustrates an apnometer 1410 according to an example embodiment. The apnometer 1410 includes a connection point 1416 for connection to the body. This may a clip on to connect to a finger or toe, or may be other connection means. A screen display 1412 presents report data and device status information to a user. As illustrated in one embodiment, the screen display 1412 presents the RDI for the entire sleep period on the left. The screen display 1412 presents the minimum SpO2 measured during the entire sleep period on the right. The apnometer 1410 also includes a power button 1414, which may be implemented with a variety of controls. For example, one push of the power button 1414 turns the power on, a second push presents the data, a third push places the device is low-power mode for monitoring a user at sleep, and a fourth push presents the sleep period data.

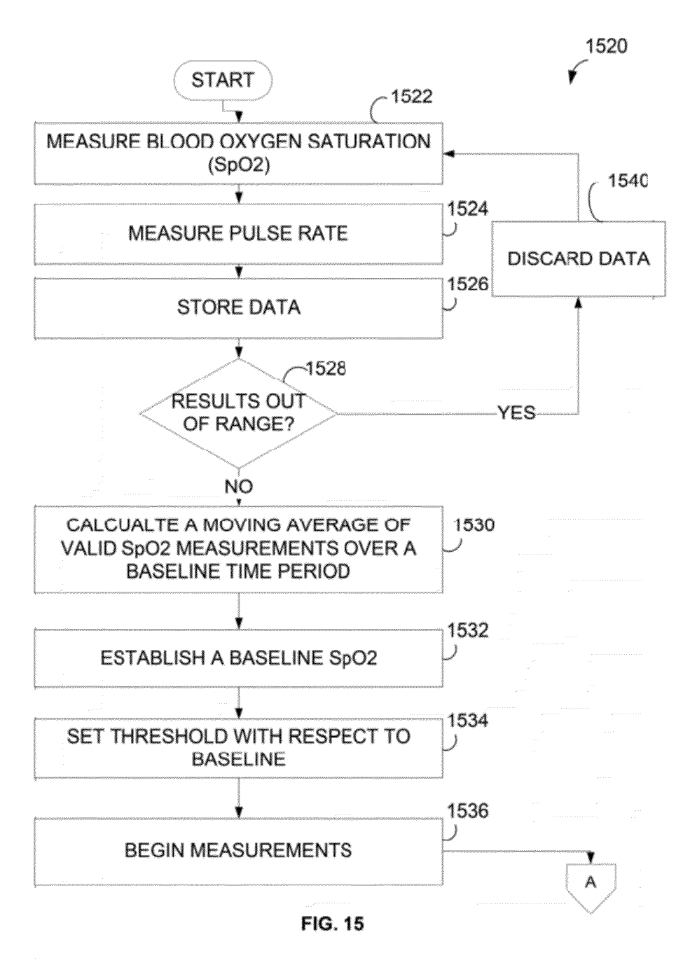

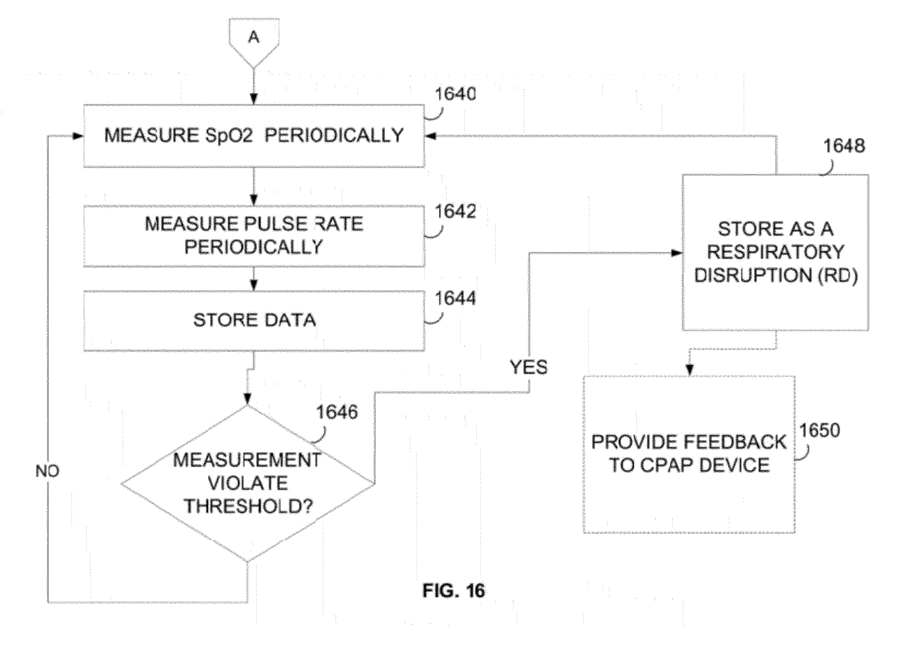

FIGS. 15 and 16 illustrate methods for operating an apnometer, according, to an example embodiment. The user places the apnometer on their body, such as on the finger. The process 1520 starts by operations to measure the blood oxygen saturation SpO2, operation 1522. The process 1520 also includes operations to measure the pulse rate, operation 1524. These measures are performed periodically, according to a sample rate and then the data is stored in memory, operation 1526. The process 1520 has operations to then compare each measured data point to threshold values and determine if the results are out of a predetermined range of values, decision point 1528. When the measurements arc out of range, the data is discarded, operation 1540; else, the process 1520 performs operations to calculate a baseline value for the SpO2 from a running or moving average of values, operation 1530. The average is taken over a baseline time period. At operation 1532, a baseline SpO2 value is determined, and a threshold set with respect to the baseline, operation 1534. The process 1520 then includes operations to begin measurements, operation 1536.

FIG. 16 illustrates operation of an apnometer, such as apnometer 10, after the initial set up procedure is complete and the thresholds and baseline value have been set. The apnomater 10 is configured to count the respiratory disruption events. The apnometer 10 turns the number of RDI events into an RDI score. The RDI score is the number of events in a given time period, such as in a 60 minute period. The apnometer 10 may use a variety of criteria in addition to those described in the present embodiment, for example, the process may consider temperature, or other measures, identifying their impact on the quality of respiration. In one embodiment, the apnometer 10 tracks the worst RDI score period of the night. By identifying the time during sleep when respiration degrades, a physician or therapist may he able to craft or revise a therapy for treatment of apnea.

In one embodiment, the apnometer 10 presents a variety of report information, including the worst time period. In one embodiment, data is tracked over a rolling window, along with the SpO2 measurements taken during that window.

The RDI calculation process implements a simplified technique which uses those inputs available on a conventional pulse oximeter. For example, the process 1520 does not require measurement of breathing airflow, and does not need to detect if the user is asleep. These simplifications are possible as the goal of the apnometer is to measure those symptoms that are consistent with sleep apnea, and provide these as an auxiliary diagnostic. In this way, it is sufficient to report the number of respiratory disruptions in the time period when respiration was worst, and this may be done while the user is wearing the diagnostic tool, i.e., the apnometer. Respiratory disruptions are rare in people who are awake, and can be reliably detected with oximetry alone.

In practice, a generic pulse oximeter may be used to measure a users blood oxygen saturation (SpO2) and pulse rate. The SpO2 measurement is a percentage of oxygen in the blood, wherein a typical value for a healthy individual experiencing no respiratory distress while awake is at least 92%. lithe apnometer reports abnormal results then it is assumed that the pulse oximeter is not properly connected to the finger, and the data should be discarded. Abnormal results include, for example, SpO2 readings less than 70%. Similarly, a pulse reading below 35 beats per minute or in excess of 200 beats per minute is considered abnormal.

At this initial stage, which may be considered a preliminary or set up stage, the ‘base line’ SpO2 is calculated, such as at operation 1532 of FIG. 15. The baseline may be determined by taking a moving average of valid SpO2 measurements over a given time period, for example over 200 seconds. A threshold level is then defined with respect to the baseline, such as four points below the baseline, as in operation 1534 of FIG. 15.

A respiratory disruption may then he defined as any event or measurement point where the SpO2 measurement is equal to or below the threshold for a minimum time period, such as for at least 10 seconds. The minimum time period t_{f} begins when the SpO2 measurements are above the threshold for at least a given satisfactory time period tₛ, such as 10 seconds. In this way, the process 1520 monitors the SpO2 measurements and when the measurements exceed the threshold for tₛ followed by measurements at or below the threshold for tᵣ, a respiratory disruption is logged.

RD=(SpO2≦Threshold)_{for tf} AND (SpO2≦Threshold)_{for ts}, wherein tₛ is the period immediately preceding t_{f}. Equ. (1)

The process 1520 then includes operations to begin measurements, operation 1536. The process 1520 continues to FIG. 16 to measure the SpO2 periodically, operation 1640, and measure the pulse rate, operation 1642. Data is stored, operation 1644. At decision point 1646 the measurement is compared to the threshold, and if the measurement exceeds the threshold, the information is stored as a respiratory disruption, operation 1648. Else, the measurements continue. In some embodiments the respiratory disruptions provide feedback for control of a CPAP device, optional operation 1650.

The apnometer 1410 in one embodiment maintains a history of respiratory measurements. In one example, this is recorded as a list or log which includes each respiratory disruption, along with the time of the disruption. The record may cover multiple time periods, or a time period defined for observation, or may identify the worst performance period and record that period. For example, the monitoring may be done over an 8 hour sleep cycle. The monitoring window length is set for 60 minutes, which is a rolling window. In this way, the monitoring identifies each 60 minute period during which the number of respiratory disruptions is a maximum. The 60 minute period is a rolling window. When such a 60 minute period is identified, the apnometer 1410 will record that time period along with measurement statistics corresponding to that time period. As monitoring continues, if there is a subsequent 60 minute time period having a higher number of respiratory disruptions, then the latter period data will overwrite the earlier period data. The time period may start at any time. In this way, the apnometer 1410 counts the number of respiratory disruptions seen in the previous 60 minutes. The count of respiratory disruptions experienced in each 60 minutes may rise and fall through the night. The highest value of the number of respiratory disruptions experienced is then reported as RD1 on the display screen 1412 of the apnometer 1410.

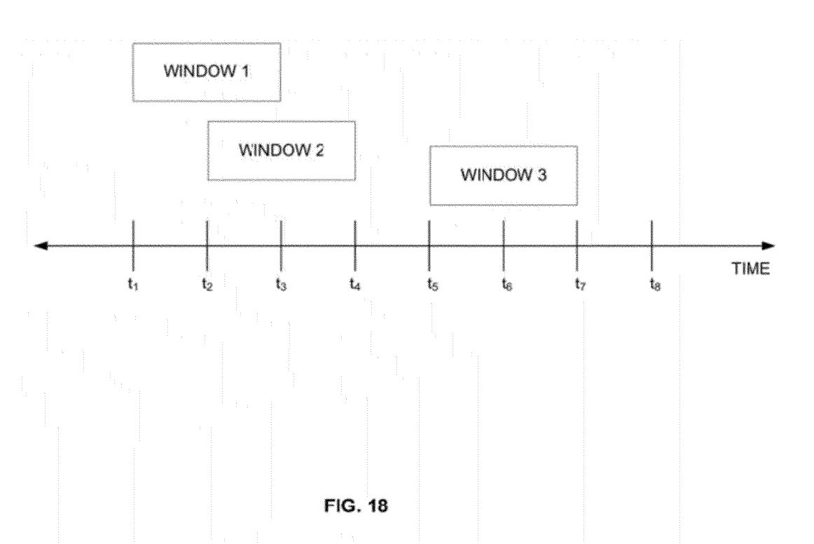

FIG. 18 illustrates various time windows identifying periods of high numbers of respiratory disruptions. The horizontal axis represents time and a first Window 1 starts at time t₁ and continues to time t₃. During Window 1 the number of respiratory disruptions is a maximum. Therefore, the Window 1 is recorded in the monitored history. As monitoring continues, during the time period from time t₂ to time t₄ a higher number of respiratory disruptions is experienced; this time period is Window 2. As Window 2 has worse performance than Window 1, the apnometer 10 stores Window 2 as the highest period of respiratory disruption. The apnometer 10 may discard the Window 1 data, or may store it for historical analysis. As monitoring continues, there is even worse performance during Window 3, which starts at time t₅ and ends at time t₇. At this point, Window 3 is stored as the worst time period, and the other data may he either maintained for historical purposes or discarded.

In some situations it may be desirable to meet the specifications or requirements that specify ease-of-use and other criteria. Consistent with such specifications, indicators may be provided to give the user feedback on the correct use and interpretation of the data measured and calculated by the apnometer 10. These are to assist the user in ensuring that the use and interpretation of the data is coffect.

In one embodiment, the screen display 1412 displays a heart heat symbol i approximating the pulse seen by the apnometer, and indicates to the user that the apnometer 1410 is properly attached to the finger. For example, when the apnometer 1410 is not able to detect a pulse, no pulsing heart will be shown on the display 12.

In some embodiments, a Time-On-Finger (TOF) indicator may be presented on the display 1412 indicating the amount of time, such as in hours and minutes, over which the apnometer detects a valid pulse and a valid SpO2 reading. This helps the user determine that the apnometer 1410 was properly attached and functioning while the user was asleep.

The apnometer has a button 1414 used to perform several functions required for simple RDI diagnostics. In a first scenario, the button 1414 is used to turn the apnometer 1410 off, and power down. This conserves battery power and allows the user to reset the device between tests. In one embodiment, the button 1414 has a 5 second hold time to implement this function. The button 14 may also be used to turn the apnometer 1410 on, such as a short press for 1 or 2 seconds. Additionally, the button 1414 may be used to toggle the screen display 1412 on and off, conserve battery power and reduce in-room light during sleep, this is also a short press for 1 or 2 seconds, wherein the sequence of selections indicates the action.

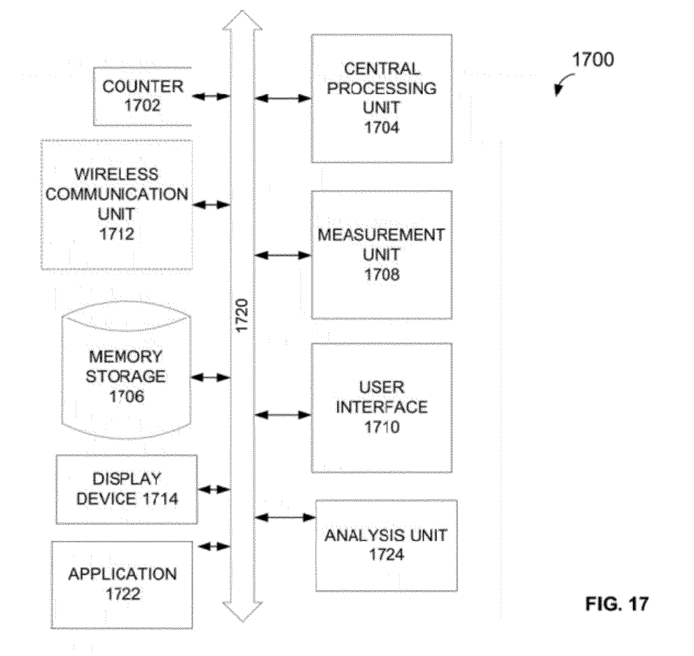

FIG. 17 illustrates a block diagram of an apnometer 1700 having a communication bus 1720 to facilitate communication between modules and units of the apnometer 1700. The communications bus 1720 is coupled to a central processing unit 1704, which implements commands and software for receiving measurement values, performing calculations on the received data, storing information in memory storage 1706, and controlling display of information. The apnometer 1700 further includes a user interface 1710 to present information to the user on a screen display 1412. The user interface may be the controller for controlling a separate screen display or may provide information to display device 1714. A measurement unit 1704 receives SpO2 information and pulse rate information from the user. The measured data is then provided to the central processing unit 1704 for further calculation, analysis and decision making. A counter 1702 allows the apnometer 1410 to keep track of the number of respiratory disruptions.

In some embodiments a wireless communication unit 1712 is provided to send information to and from the apnometer 1410. For example, the apnometer 1410 may send information to a hospital server or doctor's office. The apnometer 1410 may provide information to a CPAP unit that is configured to receive the measurement data and apply it to operation of the CPAP. Similarly, the apnometer 1410 may receive commands from external to the apnometer 1700, wherein the commands instruct as to monitor timing, monitored information, maintenance of monitored information, and so forth.

In one embodiment, the apnometer 1700 is an over-the-counter meter for the detection of sleep apnea, and may be used when someone suspects they have sleep apnea based on some symptoms associated with sleep apnea, such as snoring, daytime sleepiness, obesity, high blood pressure, heart disease, stroke, and so forth.

As illustrated in FIG. 17, the apnometer 1700 includes application 1722 for facilitating operations of the apnometer 1700. The application 1722 may be software, firmware, hardware, or a combination thereof. In some embodiments, the apnometer 1700 assist users who are using a CPAP device to monitor the devices effectiveness and/or to self-titrate the CPAP for most effective treatment. An analysis unit 1724 receives the measurement values and calculates RDI and any other indexes or indicators of respiratory performance. The analysis unit 1724 may implement any of a variety of algorithms to find the RDI corresponding to the measurement values. The analysis unit 1724 may determine the threshold values to determine a respiratory disruption, and interfaces with the counter 1702 to keep track of the respiratory disruptions that exceed the threshold.

Data is stored in the memory storage 1706 and may be retrieved for generating a report. Reported information, as well as status information, is presented on the display device 1714. A user interface 1710 is adapted to receive information from the user and display information to the user. In this way, a user may input specifics related to the measurements, such as a time period for measurement, recurring information, such as to measure each evening starting at a set time, and may also include communication information, such as where to send reports to a doctor. The user interface 1710 may be controlled or impacted by software and application data stored in applications 1722. The applications 1722 may include software to monitor sleep data, algorithms for analysis, baseline or threshold calculation and other information. The applications 1722 may access information stored in a table, such as a look up table, and compare the table information to measurements. Optionally, the apnometer 1700 may further include a wireless communication unit 1712 to send, and receive information from the apnometer.

There are a variety of implementations and configurations that may be used to provide an apnometer. There may he additional units implemented by hardware or software to add functionality and features that work in coordination with the SpO2 measuring processes. The apnometer allows for in situ calculation of parameters, as the measurement data is not only stored in a memory storage unit within the apnometer, but applications are provided to calculate parameters, such as the RDI, and then compare these to identify readings or measurements that are Out of an acceptable range of values. The applications further calculate these parameters and indexes over a rolling window, keeping cumulative scores that are time-stamped. The apnometer 10 provides a stand-alone measurement and analysis unit that does not require wires for connection, or any other external connections to modules or units. The entire device may be self-contained and thus easy to use, and causing minimal disturbance during sleep. The apnometer is a non-intrusive, easy to use device.

The apnometer 10 is a device for measuring the symptoms of sleep apnea, such as indicated by snoring and daily sleepiness during the day time. The apnometer provides an analysis and measurement tool effective in therapy for obstructive sleep apnea. The severity of a user's sleep apnea may be detected by measuring an RDI during sleep and analyzing the RDI and patterns associated therewith to determine how often breathing is interrupted during sleep.

In one embodiment, the apnometer 10 has a battery power source for user convenience. When the battery level is low, the display may present a battery symbol or the device may not turn on. There is a battery symbol visible on the display, you just recharge or replace the batteries before using the device for a sleep test. Using a single button on the apnometer the user is able to turn on the device. Pressing the button again will turn on, or turn off the display, however the device will remain on. In this example embodiment, when the device is first turned on, the display shows a‘0’ for RDI and ‘0:00’ for TOF. Any faults at start up may he corrected or cleared by holding the button down for a certain time period, such as 5 seconds. This operation acts to both reset the device and turn it on depending on the scenario.

The display will present a blinking heartbeat icon to indicate the device is properly attached. If the device happens to fall off the user's linger or otherwise become detached during the night, the user may simply reattach the device, i.e., put it back on the body. A user may try different fingers to achieve a maximum comfort during the night.

The longer the device is monitoring respiration, the more consistent the results may be, and therefore, in some embodiments, the user should set the monitoring for least four hours of sleep before accessing and relying on the statistics. The device may then be reset before repeating the test and monitoring on another night. The following table identifies the results and provides guidance to the user as to their significance and use.

Although an embodiment has been described with reference to specific example embodiments, it may he evident that various modifications and changes may be made to these embodiments without departing from the broader spirit and scope of the present discussion. Accordingly, the specification and drawings are to he regarded in an illustrative rather than a restrictive sense. The accompanying drawings that form a part hereof, show by way of illustration, and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein, individually and/or collectively, by the term “invention” merely for convenience and without intending to voluntarily limit the scope of this application to any single invention or inventive concept if more than one is in fact disclosed. Thus, although specific embodiments have been illustrated and described herein, it may be appreciated that any arrangement calculated to achieve the same purpose may he substituted for the specific embodiments shown. This disclosure is intended to cover any and all adaptations or variations of various embodiments. Combinations of the above embodiments, and other embodiments not specifically described herein, may he apparent to those of ordinary skill in the art upon reviewing the above description.

What is claimed is:

1. An airflow apparatus, comprising:
an impeller to generate an airflow at a positive applied pressure to a nasal attachment; and
a controller coupled to the impeller, the controller to receive operational information from the impeller to determine a value of the positive applied pressure, the controller to apply an adjustment policy to the impeller in response to the operational information.

2. The airflow apparatus of claim 1, wherein the operational information includes a measurement value of current supplying the impeller.

3. The airflow apparatus of claim 1, wherein the adjustment policy is to maintain the positive applied pressure at a pressure setpoint.

4. The airflow apparatus of claim 1, wherein the adjustment policy is to maintain a laminar air flow.

5. The airflow apparatus of claim 1, further comprising:
a nasal attachment; and
an attach unit coupling the nasal attachment to the impeller.

6. The airflow apparatus of claim 6, wherein the attach unit is a spring.

7. The airflow apparatus of claim 6, further comprising:
a splitter coupled to an output of the impeller; and
a pair of hoses, wherein a first hose of the pair of hoses is coupled from a first side of the splitter to a first side of the nasal attachment and a second hose of the pair of hoses is coupled from a second side of the splitter to a second side of the nasal attachment.

8. The airflow apparatus as in claim 1, further comprising:
a nasal attachment:
a moisture extractor coupled to the nasal attachment;
a moisture reservoir coupled to the moisture extractor; and
a vaporizer coupled to the moisture reservoir.

9. A method for controlling airflow generation, comprising:
receiving operational information corresponding to a positive applied pressure of an airflow generated by an impeller;
determining the positive applied pressure based on the operational information;
comparing the positive applied pressure to a pressure setpoint;
when the positive applied pressure does not equal the pressure setpoint applying an adjustment policy to the airflow generation.

10. An apparatus, comprising:
a measurement unit to measure oxygenation of blood;
an analysis unit to calculate a Respiratory Disruption Index (RDI) periodically; and
a counter to count a number of respiratory disruptions, wherein a respiratory disruption is detected when the measured oxygenation of blood is below a set point.

11. The apparatus of claim 10, wherein the analysis unit compares the number of respiratory disruptions during a given time period to a threshold value.

12. The apparatus of claim 11, wherein analysis unit calculates the threshold value.

13. The apparatus of claim 10, wherein the measurement unit measures the blood oxygenation as an SpO2 measurement.

14. The apparatus of claim 13, wherein the measurement unit measures the pulse rate.

15. The apparatus of claim 14, wherein the measurement unit measures the temperature of a user.

16. The apparatus of claim 13, wherein the analysis unit compares the blood oxygenation to a set point.

17. The apparatus of claim 16, wherein when the blood oxygenation is below the set point, the counter increments.

18. The apparatus of claim 10, wherein the apparatus has a connection mechanism for connection to a human body.

19. The apparatus of claim 10, wherein the analysis unit is to calculate a window of measurements, and identify a first window of time having a high count of respiratory disruptions.

20. The apparatus of claim 19, wherein the analysis unit is to determine a rolling window.
